SEQUENCE LISTING (65 sequences)

SEQ ID NO: 1 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XYRYRQX

SEQ ID NO: 2 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XRYRQRX

SEQ ID NO: 3 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XRYSQRX

SEQ ID NO: 4 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XFRQRQX

SEQ ID NO: 5 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XRQRQRX

SEQ ID NO: 6 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XFRQRGX

SEQ ID NO: 7 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XQRQRQX

SEQ ID NO: 8 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XYKYRQX

SEQ ID NO: 9 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XQYRQRX

SEQ ID NO: 10 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XYRQTRX

SEQ ID NO: 11 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XLRYRQX

SEQ ID NO: 12 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XYRQSRX

SEQ ID NO: 13 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XYQRQRX

SEQ ID NO: 14 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XRYTQRX

SEQ ID NO: 15 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XRFSQRX

SEQ ID NO: 16 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XQRQTRX

SEQ ID NO: 17 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XWQRQRX

SEQ ID NO: 18 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XPRYRQX

SEQ ID NO: 19 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XAYRQRX

SEQ ID NO: 20 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XVRYRQX

SEQ ID NO: 21 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XVRQRQX

SEQ ID NO: 22 - protein, 7 aa, Artificial Sequence
  Motif for Q-tag
  misc_feature (1)..(1): Xaa can be any naturally occurring amino acid
  misc_feature (7)..(7): Xaa can be any naturally occurring amino acid
XYRQRAX

SEQ ID NO: 23 - protein, 262 aa, Kutzneria albida
MHKWFLRAAVVAAVGFGLPTLIATTAQAAAVAAPTPRAPLAPPLAEDRSYRTWRVEDYVEAWERYHGREMTEDERENLARGCIGVTVVNLNREDLSNPPLNLSFGSLRTAEAVQAALNKIVDTHPSPAQYEAAVAKDPILKRLKNVVKALPSWIDSAKLKASIFSKRFYSWQNPDWSEERAHTTYRPDRETDQVDMSTYRYRARPGYVNFDYGWFDQDTNTWWHANHEEPRMVVYQSTLRHYSRPLQDFDEQVFTVAFAKKD

SEQ ID NO: 24 - protein, 196 aa, Escherichia coli
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 25 - protein, 176 aa, unknown
  Cyclobacteriaceae bacterium
MRFQSQLVFMEISENTVVGLTYELKVTNNEEDSIPFSVEVRDEEDPFYFVFGNSGLPEKFERLLENKVAGNTFNFTLSIEEAYGHADEELILTVPKKQFTGEKGFEPEMLEEGNFLPLIDEDGYPMQAKVIKDLGEELLLDFNHPLVGMNLHFDGEVYKVRKATKEETEKGYIEVN

SEQ ID NO: 26 - protein, 176 aa, unknown
  Bacteroidales bacterium
MKIGTNKVVSLAYTLEVEGDVMETVTSEKPLEFIFGTGYLLPKFEENVSNKVVGDAFDFTLTASEGYGEENPDAIIELPKDIFKVDGKIEEGLLTVGNILPMQDSDGNRLQGSIDEIKDDVVVMNFNHPLAGADLHFKGIVVAVREASETELVNGLRGELGTSCGDGGCSGCSGCH

SEQ ID NO: 27 - protein, 165 aa, Escherichia coli
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHD

SEQ ID NO: 28 - protein, 149 aa, Escherichia coli
MSESVQSNSAVLVHFTLKLDDGTTAESTRNNGKPALFRLGDASLSEGLEQHLLGLKVGDKTTFSLEPDAAFGVPSPDLIQYFSRREFMDAGEPEIGAIMLFTAMDGSEMPGVIREINGDSITVDFNHPLAGQTVHFDIEVLEIDPALEA

SEQ ID NO: 29 - protein, 149 aa, Thermus thermophilus
MKVGQDKVVTIRYTLQVEGEVLDQGELSYLHGHRNLIPGLEEALEGREEGEAFQAHVPAEKAYGPHDPEGVQVVPLSAFPEDAEVVPGAQFYAQDMEGNPMPLTVVAVEGEEVTVDFNHPLAGKDLDFQVEVVKVREATPEELLHGHAH

SEQ ID NO: 30 - protein, 7 aa, Artificial Sequence
  Motif for Q-tag
GYRYRQG

SEQ ID NO: 31 - protein, 7 aa, Artificial Sequence
  Motif for Q-tag
GRYRQRG

SEQ ID NO: 32 - protein, 7 aa, Artificial Sequence
  Motif for Q-tag
GRYSQRG

SEQ ID NO: 33 - protein, 7 aa, Artificial Sequence
  Motif for Q-tag
GFRQRQG

SEQ ID NO: 34 - protein, 7 aa, Artificial Sequence
  Motif for Q-tag
GRQRQRG

SEQ ID NO: 35 - protein, 7 aa, Artificial Sequence
  Motif for Q-tag
GFRQRGG

SEQ ID NO: 36 - protein, 7 aa, Artificial Sequence
  Motif for Q-tag
GQRQRQG

SEQ ID NO: 37 - protein, 7 aa, Artificial Sequence
  Motif for Q-tag
GYKYRQG

SEQ ID NO: 38 - protein, 7 aa, Artificial Sequence
  Motif for Q-tag
GQYRQRG

SEQ ID NO: 39 - protein, 7 aa, Artificial Sequence
  Motif for Q-tag
GYRQTRG

SEQ ID NO: 40 - protein, 7 aa, Artificial Sequence
  Motif for Q-tag
GLRYRQG

SEQ ID NO: 41 - protein, 7 aa, Artificial Sequence
  Motif for Q-tag
GYRQSRG

SEQ ID NO: 42 - protein, 7 aa, Artificial Sequence
  Motif for Q-tag
GYQRQRG

SEQ ID NO: 43 - protein, 7 aa, Artificial Sequence
  Motif for Q-tag
GRYTQRG

SEQ ID NO: 44 - protein, 7 aa, Artificial Sequence
  Motif for Q-tag
GRFSQRG

SEQ ID NO: 45 - protein, 7 aa, Artificial Sequence
  Motif for Q-tag
GQRQTRG

SEQ ID NO: 46 - protein, 7 aa, Artificial Sequence
  Motif for Q-tag
GWQRQRG

SEQ ID NO: 47 - protein, 7 aa, Artificial Sequence
  Motif for Q-tag
GPRYRQG

SEQ ID NO: 48 - protein, 7 aa, Artificial Sequence
  Motif for Q-tag
GAYRQRG

SEQ ID NO: 49 - protein, 7 aa, Artificial Sequence
  Motif for Q-tag
GVRYRQG

SEQ ID NO: 50 - protein, 7 aa, Artificial Sequence
  Motif for Q-tag
GVRQRQG

SEQ ID NO: 51 - protein, 7 aa, Artificial Sequence
  Motif for Q-tag
GYRQRAG

SEQ ID NO: 52 - protein, 117 aa, Artificial Sequence
  Q-tag example
MKVGQDKVVTIRYTLQVEGEVLDQGELSYLHGHRNLIPGLEEALEGREEGEAFQAHVPAEKAYGAGSGGGGRYRQRGGGGGSSGKDLDFQVEVVKVREATPEELLHGHAHHHHHHHH

SEQ ID NO: 53 - protein, 6 aa, Artificial Sequence
  K-tag example sequence
RYESKG

SEQ ID NO: 54 - protein, 149 aa, Artificial Sequence; identical to SEQ ID NO: 29
  SlyD amino acid sequence

SEQ ID NO: 55 - protein, 110 aa, Artificial Sequence
  SlyD with a MTG Q-tag
MKVGQDKVVTIRYTLQVEGEVLDQGELSYLHGHRNLIPGLEEALEGREEGEAFQAHVPAEKAYGAGSGGGGDYALQGGGGGSSGKDLDFQVEVVKVREATPEELLHGHAH

SEQ ID NO: 56 - protein, 110 aa, Artificial Sequence
  SlyD with a KalbTG Q-tag
MKVGQDKVVTIRYTLQVEGEVLDQGELSYLHGHRNLIPGLEEALEGREEGEAFQAHVPAEKAYGAGSGGGGYRYRQGGGGGSSGKDLDFQVEVVKVREATPEELLHGHAH

SEQ ID NO: 57 - protein, 149 aa, Artificial Sequence; identical to SEQ ID NO: 28
  SlpA amino acid sequence

SEQ ID NO: 58 - protein, 189 aa, Artificial Sequence
  main HTLV antigen and viral envelope glycoprotein amino acid sequence ′gp21′
IVSSACNNSLILPPFSLSPVPTVGSRSRRAVPVAVWFVSALAMGAGVAGGITGSMSLASGKSLLHEVDKDISQLTQAIVKNHKNLLKIAQYAAQNRRGLDLLFWEQGGLCKALQEQCCFLNITNSHVSILQERPPLENRVLTGWGLNWDLGLSQWAREALQTGITLVALLLLVILAGPCIRCPCRTMHP

SEQ ID NO: 59 - protein, 109 aa, Artificial Sequence
  amino acid sequence of modified the ′gp21′ ectodomain polypeptide sequence, engineered for better solubility, stability and reactivity in the immunoassay
MSLASGKSLLHEVDKDISQLTQAIVKNHKNLLKIAQYAAQNRRGLDLLFWEQGGLAKALQEQAAFLNITNSHVSILQERPPLENRVLTGWGLNWDLGLSQWAREALQTG

SEQ ID NO: 60 - protein, 255 aa, Artificial Sequence
  recombinant fusion protein ′TtSlyQD-Xa-gp21-8H′
MKVGQDKVVTIRYTLQVEGEVLDQGELSYLHGHRNLIPGLEEALEGREEGEAFQAHVPAEKAYGAGSGGGGDYALQGGGGGSSGKDLDFQVEVVKVREATPEELLHGHAHGGGSGGGSGGGSGGGSGGGSGGGIEGRMSLASGKSLLHEVDKDISQLTQAIVKNHKNLLKIAQYAAQNRRGLDLLFWEQGGLAKALQEQAAFLNITNSHVSILQERPPLENRVLTGWGLNWDLGLSQWAREALQTGGHHHHHHHH

SEQ ID NO: 61 - protein, 294 aa, Artificial Sequence
  recombinant fusion protein ′TtSlyD-Xa-gp21-8H′
MKVGQDKVVTIRYTLQVEGEVLDQGELSYLHGHRNLIPGLEEALEGREEGEAFQAHVPAEKAYGPHDPEGVQVVPLSAFPEDAEVVPGAQFYAQDMEGNPMPLTVVAVEGEEVTVDFNHPLAGKDLDFQVEVVKVREATPEELLHGHAHGGGSGGGSGGGSGGGSGGGSGGGIEGRMSLASGKSLLHEVDKDISQLTQAIVKNHKNLLKIAQYAAQNRRGLDLLFWEQGGLAKALQEQAAFLNITNSHVSILQERPPLENRVLTGWGLNWDLGLSQWAREALQTGGHHHHHHHH

SEQ ID NO: 62 - protein, 427 aa, Artificial Sequence
  recombinant fusion protein ′TtSlyKQD-SlpA-Xa-gp21-8H′
MKVGQDKVVTIRYTLQVEGEVLDQGELSYLHGHRNLIPGLEEALEGREEGEAFQAHVPAEKAYGAGSGGGGYRYRQGGGGGSSGKDLDFQVEVVKVREATPEELLHGHAHGGGSGGGSGGGSGGGSGGGSGGGMSESVQSNSAVLVHFTLKLDDGTTAESTRNNGKPALFRLGDASLSEGLEQHLLGLKVGDKTTFSLEPDAAFGVPSPDLIQYFSRREFMDAGEPEIGAIMLFTAMDGSEMPGVIREINGDSITVDFNHPLAGQTVHFDIEVLEIDPALEAGGGSGGGSGGGSGGGSGGGSGGGIEGRMSLASGKSLLHEVDKDISQLTQAIVKNHKNLLKIAQYAAQNRRGLDLLFWEQGGLAKALQEQAAFLNITNSHVSILQERPPLENRVLTGWGLNWDLGLSQWAREALQTGGHHHHHHHH

SEQ ID NO: 63 - protein, 24 aa, Artificial Sequence
  tryptic peptide ,62-85′
AYGAGSGGGGDYALQGGGGGSSGK

SEQ ID NO: 64 - protein, 28 aa, Artificial Sequence
  tryptic peptide ,241-255′
ALQEQAAFLNITNSHVSILQERPPLENR

SEQ ID NO: 65 - protein, 15 aa, Artificial Sequence
  tryptic peptide ,241-255′
EALQTGGHHHHHHHH

(19) United States
(12) Patent Application Publication
(10) Pub. No.: US 2019/0381181 A1
(43) Pub. Date: Dec. 19, 2019

(54) FKBP DOMAIN WITH TRANSGLUTAMINASE RECOGNITION SITE

(71) Applicant: Roche Diagnostics Operations, Inc., Indianapolis, IN (US)

(72) Inventors: Michael Schraeml, Penzberg (DE); Wojtek Steffen, Penzberg (DE)

(73) Assignee: Roche Diagnostics Operations, Inc., Indianapolis, IN (US)

(21) Appl. No.: 16/008,281

(22) Filed: Jun. 14, 2018

Related U.S. Application Data
(63) Continuation of application No. PCT/EP2016/080847, filed on Dec. 13, 2016.

(30) Foreign Application Priority Data
Dec. 15, 2015 (EP) 15200111.1

(51) Int. Cl. A61K 47/65; C07K 1/13; C12N 9/90

(52) U.S. Cl. CPC A61K 47/65; C07K 1/13; C07K 2319/35; C07K 2319/21; C12N 9/90

(57) ABSTRACT

The present disclosure relates to a recombinant transglutaminase (TG) substrate having an amino acid sequence of the FKBP domain of an FKBP polypeptide, wherein the “insert-in-flap” (IF) domain thereof is, at least in part, replaced by an amino acid sequence (“Q-tag”) of 5 to 20 amino acids with a sequence having at least 80% sequence identity to the YRYRQ portion of the peptide sequence X₁-YRYRQ-X₂ (SEQ ID NO. 1), and wherein said TG substrate is a substrate for the TG function of the Kutzneria albida TG. The present disclosure furthermore relates to uses of said substrate.

### CROSS-REFERENCE TO RELATED APPLICATIONS

This application is a continuation of International Application No. PCT/EP2016/080847 filed Dec. 13, 2016, which claims priority to European Application No. 15200111.1 filed Dec. 15, 2015, the disclosures of which are hereby incorporated by reference.

### BRIEF DESCRIPTION OF THE INVENTION

The present invention relates to a recombinant transglutaminase (TG) substrate comprising an amino acid sequence of the FKBP domain of an FKBP polypeptide, wherein the “insert-in-flap” (IF) domain thereof is, at least in part, replaced by an amino acid sequence (“Q-tag”) of 5 to 20 amino acids comprising a sequence having at least 80% sequence identity to the peptide sequence X₁-YRYRQ-X₂ (SEQ ID NO. 1), specifically to the YRYRQ portion of the peptide sequence, and wherein said TG substrate is a substrate for the TG function of the Kutzneria albida TG (KalbTG). The present invention furthermore relates uses of said substrate.

### BACKGROUND OF THE INVENTION

Soluble and/or immunoreactive antigens and variants thereof are essential for in vitro diagnostic tests. As an example, a recombinant and soluble variant of the viral coat protein gp41 is used in order to detect an HIV-1 infection.

The required solubility of immunoreactive antigens and variants thereof can be a challenge in the design of effective assays, but can be improved by fusing to one or more chaperone units having peptidyl prolyl isomerase (PPIase) activity. The technique of using a protein scaffold for engineering polypeptide domains displayed by the scaffold is known in the field of antibodies and antibody fragments. Thus, domains such as variable loops of antigen binding regions of antibodies have been extensively engineered to produce amino acid sequence segments having improved binding (e.g. affinity and/or specificity) to known targets (see WO 2014/071978).

FK506 binding proteins (FKBPs) have been identified in many eukaryotes from yeast to humans and function as protein folding chaperones for proteins containing proline residues. Along with cyclophilin, FKBPs belong to the immunophilin family. In the human genome there are encoded fifteen proteins whose segments have significant homology with the sequence of 12 kDa protein which is the target of the potent immunosuppressive macrolides FK506 or rapamycin. The 12 kDa archetype of the FK506-binding protein (FKBP), known as FKBP-12, is an abundant intracellular protein. FKBP12 functions as a PPIase that catalyzes interconversion between prolyl cis/trans con-formations. FKBPs are involved in diverse cellular functions including protein folding, cellular signaling, apoptosis and transcription. They elicit their function through direct binding and altering conformation of their target proteins, hence acting as molecular switches.

The bacterial slyD gene encodes a FKBP-type peptidyl-prolyl cis-trans isomerase (PPIase). SlyD is a bacterial two-domain protein that functions as a molecular chaperone, a prolyl cis/trans isomerase, and a nickel-binding protein. The chaperone function located in one domain of SlyD is involved in twin-arginine translocation and increases the catalytic efficiency of the prolyl cis/trans isomerase domain in protein folding by two orders of magnitude.

Problems with the folding of the recombinant gene product as well as protein aggregation, i.e., formation of inclusion bodies, are frequently encountered in Escherichia coli. This is particularly true for proteins that carry structural disulfide bonds, including antibody fragments, cytokines, growth factors, and extracellular fragments of eukaryotic cell surface receptors. Therefore, they have developed the helper plasmid pTUM4, which effects overexpression of four established periplasmic chaperones and/or folding catalysts: the thiol-disulfide oxidoreductases DsbA and DsbC, which catalyze the formation and isomerization of disulfide bridges, and two peptidyl-prolyl cis/trans isomerases with chaperone activity, FkpA and SurA.

The E. coli SlyD and FKBP12 (wild type and mutants C23A and C23S) can be recombinantly produced in E. coli in high yield in soluble form. FKBP derived from thermophilic organisms and E. coli SlyD can be used as chaperones in the recombinant expression of chimeric polypeptides in E. coli. The E. coli SlyD and FKBP12 polypeptides are reversibly folding polypeptides. The crystal structure and functional characterization of the metallochaperone SlyD from Thermus thermophilus. Thermus thermophilus consists of two domains representing two functional units. PPIase activity is located in a typical FKBP domain, whereas chaperone function is associated with the autonomously folded insert-in-flap (IF) domain. The two isolated domains are stable and functional in solution.

SlyD is a bacterial two-domain protein that functions as a molecular chaperone, a prolyl cis/trans isomerase, and a nickel-binding protein. They summarize recent findings about the molecular enzyme mechanism of SlyD. The chaperone function located in one domain of SlyD is involved in twin-arginine translocation and increases the catalytic efficiency of the prolyl cis/trans isomerase domain in protein folding by two orders of magnitude.

The amino acid sequence of the FKBP 12 polypeptide comprises a single tryptophan residue at position 60. Thus, FKBP12 mutants can be analyzed for structural integrity simply by analyzing the tryptophan fluorescence. A test for remaining catalytic activity of the FKBP 12 mutant can be performed by determining the remaining rotamase activity. It is also possible to determine the structural integrity of FKBP 12 mutants by determining the FK506- or Rapamycin binding.

Parvulins are small prolyl isomerases that serve as catalytic domains of folding enzymes. SurA (survival protein A) from the periplasm of Escherichia coli consists of an inactive (Par1) and an active (Par2) parvulin domain as well as a chaperone domain. In the absence of the chaperone domain, the folding activity of Par2 is virtually abolished. They created a chimeric protein by inserting the chaperone domain of SlyD, an unrelated folding enzyme from the FKBP family, into a loop of the isolated Par2 domain of SurA. This increased its folding activity 450-fold to a value higher than the activity of SurA, in which Par2 is linked with its natural chaperone domain. In the presence of both the natural and the foreign chaperone domain, the folding activity of Par2 was 1500-fold increased. Related and unrelated chaperone domains thus are similarly efficient in enhancing the folding activity of the prolyl isomerase Par2. A sequence analysis of various chaperone domains suggests that clusters of exposed methionine residues in mobile chain regions might be important for a generic interaction with unfolded protein chains. This binding is highly dynamic to allow frequent transfer of folding protein chains between chaperone and catalytic domains.

For immunological diagnostic systems, antigens and/or antibodies or other immunological binding partners (proteins of interest) are furthermore often conjugated, for example with biotin (for an immobilization with streptavidin) or other labels, like ruthenium, for their detection. Usually, chemical methods are used in order to conjugate such markers to reactive amino- or sulfhydryl-moieties.

Unfortunately, conventional chemical strategies for protein modification are difficult to control and give rise to heterogeneous populations of immunoconjugates with variable stoichiometries, each of which has its own in vivo characteristics. Furthermore, the number of conjugated molecules or label can not be controlled, and thus is not defined, and usually follows a normal distribution, which causes problems when wishing to quantify reactions.

The introduction of artificial, bio-orthogonal groups for site-specific and stoichiometric protein modification offers a potential solution to this problem. Such strategies are en vogue but are often laborious and still risk product heterogeneity. Furthermore, all components of the system must be and have to remain stable over prolonged periods of time, and under conjugation conditions. Also, the position/location of the conjugation(s) can not be controlled. A non-specific inclusion of the marker, for example in or close to the active center of a protein, or at a position at or close to the position(s) that mediate immunological activities can interfere with the immune-reactivity or even fully inhibit it.

Methods for a site-specific enzymatic conjugation of markers are known (e.g. sortase, MTG), but these require the presence of an additional specific recognition sequence (tag sequence) in the protein of interest to be labelled

Microbial transglutaminase (MTG) is one of the most important enzymes for the crosslinking of proteins and peptides in many food- and biotechnological applications. MTG was first discovered in and later extracted from the organism Streptomyces mobaraensis. Recombinantly produced Streptomyces mobaraensis MTG represents the bulk of industrially used MTG today. The enzyme catalyzes the formation of an isopeptide bond between an acyl-group, e.g. a glutamine (Q) side chain and an alkyl-amine, e.g. a lysine (K) side chain. In absence of reactive amine groups, the enzymatic reaction with water leads to deamination of Glutamine side chains. The bacterial enzyme works without the addition of cofactors such as Ca²⁺ or GTP and in a broad range of pH-, buffer- and temperature conditions. MTG has already been used for the development of therapeutic Antibody-drug conjugates, but due to the low specificity of the enzyme, the large-scale production of such MTG-mediated conjugates has not yet been established. All known active microbial transglutaminase species exhibit molecular weights >38 kDa. Being a cross-linking enzyme in nature, microbial transglutaminase displays broad substrate specificity for amine-donor molecules and a relatively low specificity for acyl-donors. Since only the substrate specificities of the enzyme from Streptomyces mobaraenis and homolog enzymes are known, a bio-orthogonal conjugation approach, e.g. simultaneous labeling of a biomolecule using two or more different label-substrates and two or more transglutaminase species, is currently not possible.

### BRIEF DESCRIPTION OF THE DRAWINGS

FIG. 1A shows the amino acid sequences of the transglutaminase from Kutzneria albida (database no. WP_030111976).

FIG. 1B shows the amino acid sequence of slyD of E. coli.

FIG. 1C shows the amino acid sequence of slyD of Cyclobacteriaceae bacterium AK24.

FIG. 1D shows the amino acid sequence of slyD of Bacteroidales bacterium.

FIG. 2A shows the results of labeling using Cy3 or Cy5 according to the examples, below.

FIG. 2B shows the relative pH independence in the range of between 6.2 to 9.0.

FIG. 3A shows the results of labeling using SDS-PAGE according to the examples, below.

FIG. 3B shows the general structure of a ruthenium labelled fusion construct.

FIG. 4 shows a preferred embodiment of a substrate-labeled protein of interest (here, two chains of an antibody) according to the present invention.

FIG. 5A presents a schematic view of ‘TtSlyQD-Xa-gp21-8H’ of SEQ ID NO:60. The SlyD portions are indicated, further the Q-tag in the fusion protein, as well as a recognition site for factor Xa protease, and the ‘gp21’ portion as indicated in the examples. The star symbolizes a Ruthenium label.

FIG. 5B shows results after SDS-PAGE analysis showing specific and unspecific Ruthenium labeling of the recombinant gp21 (HTLV) antigen with the bacterial transglutaminase of Streptomyces mobaraensis. Depicted are unlabeled, single-labeled, double-labeled, and triple-labeled ‘TtSlyQD-Xa-gp21-8H’ fusion proteins after Ru labeling.

FIG. 5C depicts the Ruthenium label.

FIG. 6A shows a diagram of a ruthenium labeled construct described herein.

FIG. 6B shows results using labeled ‘TtSlyKQD-SlpA-Xa-gp21-8H’ according to SEQ ID NO:62 in an Elecsys assay.

FIG. 6C shows results using labeled ‘TtSlyKQD-SlpA-Xa-gp21-8H’ according to SEQ ID NO:62 in an Elecsys assay.

FIG. 6D shows results using labeled ‘TtSlyKQD-SlpA-Xa-gp21-8H’ according to SEQ ID NO:62 in an Elecsys assay.

FIG. 6E shows graphical results using labeled ‘TtSlyKQD-SlpA-Xa-gp21-8H’ according to SEQ ID NO:62 in an Elecsys assay.

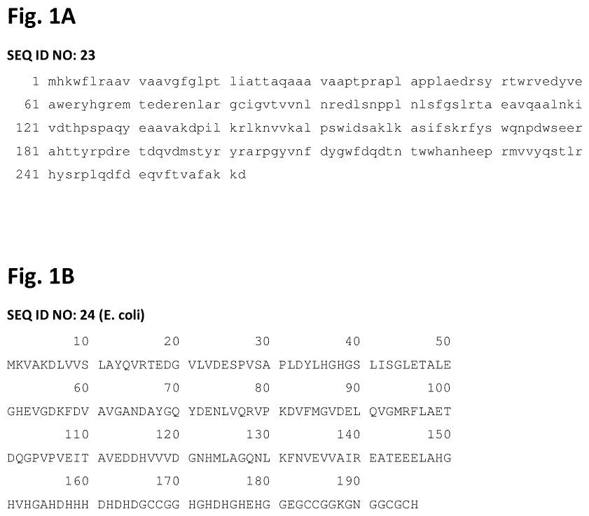

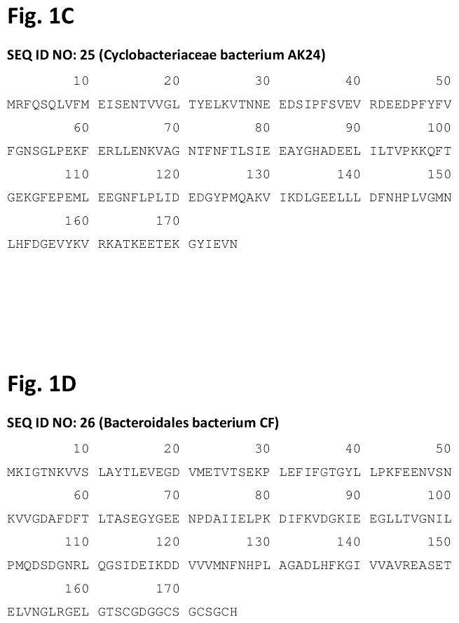

### BRIEF DESCRIPTION OF THE SEQUENCE IDENTIFIERS

SEQ ID NO: 1 to 22 show the sequences of the Q-tag motifs as identified in the context of the present invention, wherein the N-terminal X is as X₁ above, and the C-terminal X is as X₂ as above.

SEQ ID NO: 23 shows the amino acid sequence of the transglutaminase from Kutzneria albida (database no. WP_030111976).

SEQ ID NO: 24 shows the amino acid sequence of slyD of E. coli.

SEQ ID NO: 25 shows the amino acid sequence of slyD of Cyclobacteriaceae bacterium AK24.

SEQ ID NO: 26 shows the amino acid sequence of slyD of Bacteroidales bacterium CF.

SEQ ID NO: 27 shows the amino acid sequence of the FKBP domain of slyD of E. coli.

SEQ ID NO: 28 shows the amino acid sequence of the FKBP domain of FKBP16 of E. coli.

SEQ ID NO: 29 shows the amino acid sequence of the FKBP domain of slyD of Thermus thermophilus.

SEQ ID NO: 30 to 51 show the sequences of the Q-tag motifs as identified in the context of the present invention, wherein the N-terminal and C-terminal amino acid is one exemplary glycine linker.

SEQ ID NO: 52 shows the amino acid sequence of the KalbTG glutamine donor sequence that was recombinantly grafted onto the FKBP domain of SlyD.

SEQ ID NO: 53 shows the amino acid sequence of a transglutaminase lysine donor sequence (K-tag).

SEQ ID NO: 54 shows the SlyD amino acid sequence.

SEQ ID NO: 55 shows the amino acid sequence of SlyD with a MTG Q-tag.

SEQ ID NO: 56 shows the amino acid sequence of SlyD with a KalbTG Q-tag.

SEQ ID NO: 57 shows the SlpA amino acid sequence.

SEQ ID NO: 58 shows the main HTLV antigen and viral envelope glycoprotein amino acid sequence ‘gp21’.

SEQ ID NO: 59 shows the amino acid sequence of modified the ‘gp21’ ectodomain polypeptide sequence, engineered for better solubility, stability and reactivity in the immunoassay.

SEQ ID NO: 60 shows the amino acid sequence of the recombinant fusion protein ‘TtSlyQD-Xagp21-8H’.

SEQ ID NO: 61 shows the amino acid sequence of the recombinant fusion protein ‘TtSlyD-Xagp21-8H’.

SEQ ID NO: 62 shows the amino acid sequence of the recombinant fusion protein ‘TtSlyKQDSlpA-Xa-gp21-8H’.

### DETAILED DESCRIPTION OF THE INVENTION

In view of the above, it is an object of the present invention to provide new tools and methods in order to provide for an efficient and controlled labelling of antigens and/or antibodies or other immunological binding partners (proteins of interest) in the context of immunological diagnostic and/or therapeutic systems and methods. Other aspects and objects will become apparent for the person of skill upon reading the following more detailed description of the invention.

According to a first aspect thereof, the above objects are solved by the provision of a recombinant transglutaminase (TG) substrate according to the following general formula I

(F*-L)_{y}-X (I).

In said formula (I), F* is selected from an amino acid sequence of the FKBP domain of an FKBP polypeptide, preferably comprising an “insert-in-flap” (IF) domain that in the unmodified polypeptide is inserted internally as a guest into a surface loop of the host domain, which is the prolyl isomerase of the FK506 binding protein (FKBP) type. Nevertheless, the invention also includes FKBP domains that naturally (unmodified) do not include an “insert-in-flap” (IF) domain, wherein the Q-tag is inserted at the homologous position in the enzyme.

Said “insert-in-flap” (IF) domain is, at least in part, replaced by an amino acid sequence (“Q-tag”) having a length of 5 to 20 amino acids comprising a sub-sequence of 5 contiguous amino acids, the sub-sequence having at least 80% sequence identity to the peptide sequence X₁-YRYRQ-X₂ (SEQ ID NO. 1), specifically to the YRYRQ portion of the peptide sequence X₁-YRYRQ-X₂ (SEQ ID NO. 1), wherein X₁ and X₂ are absent or constitute linker amino acids. In a specific embodiment, the IF domain is, at least in part, replaced by X₁-YRYRQ-X₂ (SEQ ID NO. 1) (“Q-tag”) having a length of 5 to 15 or 5 to 20 amino acids, the Q-tag comprising a contiguous sub-sequence of five amino acids (i.e. a sub-sequence consisting of five contiguous amino acid residues), the sub-sequence having at least 80% sequence identity to the YRYRQ portion of (in) the peptide sequence X₁-YRYRQ-X₂(SEQ ID NO. 1), wherein X₁ and X₂ are absent or constitute linker amino acids.

In a more specific embodiment, only X₁ is absent and the length of the peptide sequence X₁-YRYRQ-X₂ is 6 to 15 or 6 to 20 amino acids. In an even more specific embodiment, X₂ comprises (or may consist of) an arginine residue directly following the YRYRQ sub-sequence or a subsequence having at least 80% sequence identity to the YRYRQ portion.

In yet a further very specific embodiment, in the sub-sequence consisting of five contiguous amino acid residues having at least 80% sequence identity to the YRYRQ portion of the peptide sequence X₁-YRYRQ-X₂ (SEQ ID NO. 1), a glutamine residue is on a selected position of the sub-sequence, the position in the sub-sequence being selected from the group consisting of the third position, the fourth position, the fifth position, and a combination thereof.

In yet a further very specific embodiment, in the sub-sequence consisting of five contiguous amino acid residues having at least 80% sequence identity to the YRYRQ portion of the peptide sequence X₁-YRYRQ-X₂ (SEQ ID NO. 1), an arginine residue is on a selected position of the sub-sequence, the position in the sub-sequence being selected from the group consisting of the fourth position, the fifth position, and a combination thereof.

It is further understood that, according to the invention, the linker amino acids X₁ and X₂, if present, are selected independently from each other.

Preferred are Q-tags according to the present invention that re selected from the sequences X₁-YRYRQ-X₂ (SEQ ID NO: 1), X₁-RYRQR-X₂ (SEQ ID NO: 2), X₁-RYSQR-X₂ (SEQ ID NO: 3), X₁-FRQRQ-X₂ (SEQ ID NO: 4), X₁-RQRQR-X₂ (SEQ ID NO: 5), X₁-FRQRG-X₂ (SEQ ID NO: 6), X₁-QRQRQ-X₂ (SEQ ID NO: 7), X₁-YKYRQ-X₂ (SEQ ID NO: 8), X₁-QYRQR-X₂ (SEQ ID NO: 9), X₁-YRQTR-X₂ (SEQ ID NO: 10), X₁-LRYRQ-X₂ (SEQ ID NO: 11), X₁-YRQSR-X₂ (SEQ ID NO: 12), X₁-YQRQR-X₂ (SEQ ID NO: 13), X₁-RYTQR-X₂ (SEQ ID NO: 14), X₁-RFSQR-X₂ (SEQ ID NO: 15), X₁-QRQTR-X₂ (SEQ ID NO: 16), X₁-WQRQR-X₂ (SEQ ID NO: 17), X₁-PRYRQ-X₂ (SEQ ID NO: 18), X₁-AYRQR-X₂ (SEQ ID NO: 19), X₁-VRYRQ-X₂ (SEQ ID NO: 20), X₁-VRQRQ-X₂ (SEQ ID NO: 21), and X₁-YRQRA-X₂ (SEQ ID NO: 22), wherein X₁ and X₂ are as above.

It was surprisingly found that the two parts of the FKBP domain upstream and downstream insertion of the IF domain function as structurally rather rigid and precise scaffold or “collar” for the sequence that is inserted into and/or replaces the IF domain part (“head”, “Q-tag”). Because of this, the presentation and orientation of the insertion/replacement reliably does not substantially interfere with any other function of the other components of the substrate, in particular the function(s) of the protein of interest (X). Furthermore, the presentation of the—in this case—TG substrate binding site (Q-tag) leads to a highly controlled stoichiometric binding of the label, and hence labelling of the protein of interest.

In formula (I), L is absent or is selected from a linker amino acid sequence. Preferably, said linker sequence L comprises between 1 to 20 amino acids and more preferred said amino acids do not interfere essentially with the function(s) of the FKBP domain (in particular the replacement/insertion) and/or the protein (s9 of interest (e.g. immunological functions).

X designates the protein of interest; that is preferably selected from an enzyme, an antigen, such as a viral protein, an antibody or fragment thereof, and other immunological binding partners.

In formula (I), y is an integer of between 1 and 100, thus, several marker groups F*-L can be attached to the protein of interest.

Preferably, the recombinant transglutaminase (TG) substrate according to the invention can furthermore comprise protein tags for purification and/or immobilization, for example at the N_ and/or C-terminus, like biotin, maltose or his-tags(s).

Preferably, the different components of the substrate F*, L and/or X are covalently attached with each other in order to allow the controlled labeling of the substrate using the TG activity. The substrate can be recombinantly produced as a fusion protein, and expressed and purified from hosts cells, such as, for example, bacterial or yeast host cells. Respective methods are well known to the person of skill. The components can also be produced (e.g. synthesized) separately or in parts, and are then subsequently joined, either covalently or conjugated, depending on the circumstances and the desired purpose(s) thereof.

According to the invention, the inventive TG substrate is a substrate for the transglutaminase (TG) function of the Kutzneria albida TG according to SEQ ID No. 23, or a homologous protein that is identical to at least 80%, preferably to at least 90%, more preferably to at least 95, 98 or 99% to the amino acid sequence thereof, and exhibit substantial TG activity as described herein. Preferably, the TG polypeptide or a part thereof having a substantial transglutaminase function or activity is cloned and recombinantly produced in hosts cells, and subsequently (at least in part) purified, depending on the circumstances and the desired purpose(s) thereof. Respective methods are well known to the person of skill. The TG activity can be measured using assays that are also well known to the person of skill. “Recombinant TG substrate” in the context of the invention shall mean that the substrate as a whole does not occur in nature.

The FKBP domain as used for the recombinant transglutaminase (TG) substrate according to the present invention is preferably selected from a eukaryotic or bacterial FKBP polypeptide selected from FKBP12, AIP, AIPL1, FKBP1A, FKBP1B, FKBP2, FKBP3, FKBP5, FKBP6, FKBP7, FKBP8, FKBP9, FKBP9L, FKBP10, FKBP11, FKBP14, FKBP15, FKBP52, LOC541473, and SLYD, and homologs of the FKBP domains thereof that are identical to at least 80%, preferably to at least 90%, more preferably to at least 95, 98 or 99% to the amino acid sequence thereof, and are suitable for the insertion of a Q-tag. Respective domains can be analyzed for their suitability as described herein, and in the literature, e.g. using alignment programs, in particular to identify structural alignments.

In an embodiment, a FKBP domain as used for the recombinant transglutaminase (TG) substrate according to the present invention comprises an amino acid sequence of a polypeptide with PPIase activity. The polypeptide with PPIase activity is of prokaryotic or eukaryotic origin, or the polypeptide with PPIase activity as an artificial variant (mutant) thereof. An artificial variant of a polypeptide with PPIase activity can be generated by a technique according to the state of the art of protein engineering. According to the invention, an artificial variant of a polypeptide with PPIase activity is characterized e.g. by replacement, addition or deletion of one or more amino acids. In a specific embodiment, PPIase activity and/or chaperone function of the FKBP domain as used for the recombinant transglutaminase substrate according to the present invention is/are preserved in an artificial variant.

The catalytic activity of human FKBP12 as a prolyl isomerase is high towards short peptides, but very low in proline-limited protein folding reactions. In contrast, the SlyD proteins, which are members of the FKBP family, are highly active as folding enzymes. They contain an extra “insertin-flap” or IF domain near the prolyl isomerase active site. The excision of this domain did not affect the prolyl isomerase activity of SlyD from Escherichia coli towards short peptide substrates but abolished its catalytic activity in proline-limited protein folding reactions. The reciprocal insertion of the IF domain of SlyD into human FKBP12 increased its folding activity 200-fold and generated a folding catalyst that is more active than SlyD itself. The IF domain binds to refolding protein chains and thus functions as a chaperone module.

In E. coli, it is believed that amino acids 1 to 69 of SLYD form the first part of the PPIase domain, amino acids 76 to 120 form the IF-chaperone (domain), and amino acids 129 to 151 form the second part of the PPIase domain. Thus, amino acids 1 to 151 form the FKBP-domain. Amino acids 152 to 196 are involved in metal binding.

Preferred is the recombinant transglutaminase (TG) substrate according to the present invention, wherein said FKBP domain has a length of between about 120 to 170, preferably between about 130 to 160, and most preferred between about 145 to 155 of the N-terminal amino acids of said FKBP polypeptide. This includes the IF-domain sequence.

“About” in the context of the present invention shall mean+/−10% of a given value.

Preferred is the recombinant transglutaminase (TG) substrate according to any one of claims 1 to 3, wherein said FKBP domain comprises the N-terminal amino acids 1 to 64 and 123 to 149 of the SLYD polypeptide, and wherein amino acids 65 to 122 are replaced by said Q-tag.

As mentioned above, the linker sequence L can comprise between 1 to 20 amino acids, preferably 1 to 10 amino acids, more preferred 1 to 5 amino acids wherein preferably said amino acids to not interfere essentially with the FKBP domain and/or the protein of interest. Preferred linker amino acids are small amino acids, like glycine or alanine. Amino acid linkers and their compositions are known in the art (see, e.g. Chichili et al. Linkers in the structural biology of protein—protein interactions Protein Sci. 2013 February; 22(2): 153-167).

Proteins of interest in the context of the present invention are generally all proteins that can be labelled using the present technology. Preferred is the recombinant transglutaminase (TG) substrate according to the present invention, wherein said protein of interest is selected from an enzyme, an antigen, such as a viral protein, an antibody or fragment thereof, and other immunological binding partners. The present invention is of particular use for immunological reactions and assays, where quantification is desired.

Another aspect of the present invention then relates to an in vitro method for labelling a protein of interest, comprising a) providing the recombinant transglutaminase (TG) substrate according to the present invention comprising a protein of interest; b) providing an effective amount of the transglutaminase of Kutzneria albida (e.g. according to SEQ ID NO: 23) or a homolog thereof as described herein, c) providing a suitable label comprising an alkyl-amine group (“amine donor”), such as, for example, a lysine, and d) contacting said components according to a) to c), whereby said transglutaminase (activity) attaches said label to said substrate.

Preferred is a method according to the present invention, wherein said transglutaminase of Kutzneria albida or a homolog thereof as described herein is recombinantly produced, as described herein.

In this method according to the present invention, the substrate comprising the F* group comprising the Q-tag (optionally replacing the IF-domain region) is labelled using the activity of the transglutaminase of Kutzneria albida. Preferably, the label to be attached is selected from an enzyme, biotin, a radioactive group, a dye, such as a fluorescent dye, an isotope, and a metal. Said label is part of the label component/compound that comprises alkyl-amine group, such as, for example, a lysine. Most preferred, said label component/compound comprises an amine-donor tag (“K-tag”) having at least 80% sequence identity to the peptide sequence RYESK. Examples for preferred K-tags and their composition are described below and can also be found in the literature.

Preferred is a method according to the present invention, wherein said labeling is controlled and, for example, achieved in a stoichiometric ratio of label and protein of interest, for example at about 1:1. Multiple labeling can also be achieved using additional enzymes (e.g. other TGs than KalbTG) and respective other TG-substrates, in order to attach two or even multiple labels to a protein/proteins of interest.

Preferred is the method according to the present invention, wherein said protein of interest is selected from an enzyme, an antigen, such as a viral protein, an antibody or fragment thereof, and other immunological binding partners. The present invention is of particular use for immunological reactions and assays, where quantification is desired.

The choice of the protein of interest can also depend on the intended use of the piRNA molecules as used, either in therapy, research and/or diagnosis. Preferred is the method according to the invention, wherein said protein of interest is selected from protein involved/causing in cancer, neurological diseases, immunological diseases, allergy, metabolic diseases, fertility (e.g. reproductive rate or number), animal production (e.g. amount of meat or milk), protein essential for cell growth and/or development, for mRNA degradation, for translational repression, and/or for transcriptional gene silencing.

Another aspect of the present invention then relates to a pharmaceutical or diagnostic composition comprising at least one recombinant transglutaminase (TG) substrate according to the invention, together with pharmaceutically acceptable carriers and components, such as buffers. Pharmaceutical or diagnostic compositions for multiple labeling can also be obtained that comprise additional enzymes (e.g. other TGs than Kalb-TG) and respective other TG-substrates, in order to be able to attach two or even multiple labels to a protein/proteins of interest.

Another aspect of the present invention then relates to a pharmaceutical or diagnostic composition comprising at least one labeled protein of interest as produced according to a method according to the present invention, together with pharmaceutically acceptable carriers and components, such as buffers.

Preferred is the pharmaceutical or diagnostic composition according to the present invention, wherein said protein of interest is labelled at a stoichiometric ratio of label and protein of interest of, for example, about 1:1. Two or even several labels can also be attached to a protein/proteins of interest.

Another aspect of the present invention then relates to a diagnostic kit, comprising the diagnostic composition according to the present invention, optionally together with other components for performing an immunoassay. The kit can comprise, in joint or separate containers, a microbial recombinant transglutaminase, e.g. a purified microbial transglutaminase having at least 80% sequence identity to the Kutzneria albida microbial transglutaminase as described herein. The kit may further include substrates, such as a substrate including an amine-donor tag having at least 80% sequence identity to the peptide sequence RYESK. The kit can also include instructions for performing reactions (e.g. immunoassays) that require use of the substrate according to the invention, either labeled or un-labeled.

Another aspect of the present invention the relates to the use of the recombinant transglutaminase (TG) substrate, the pharmaceutical or diagnostic composition or the kit according to the present invention for labeling or in the labeling of a protein of interest, in particular in immunological reactions and assays, where a quantification is desired.

The methods of the present invention can be performed in vivo or in vitro, in particular in laboratory animals, or in culture.

The present invention will now be illustrated further in the following non-limiting examples, with reference to the accompanying figures. For the purposes of the invention, all publications, patent applications, patents, and other references mentioned herein are incorporated by reference in their entirety.

### EXAMPLES

### Identification of the Position of the IF Domain

In principle, alignments of the primary structure of the amino acid sequences of FKBP domains can be used in order to identify the position of the IF domain to be replaced (or added), e.g. using the program Clustal Omega. An alternative, in particular with respect to non-bacterial FKBPs is the alignment of 3D structures (e.g. using the program PyMol), since the domain constitutes a structurally conserved element.

Specific examples are as follows:

SLYD_ECOLI FKBP-type peptidyl-prolyl cis-trans isomerase SlyD of Escherichia coli (strain K12). FKBP domain, IF domain double underlined.

(SEQ ID NO: 27)
MKVAKDLVVSLAYQVRTEDGLVLVDESPVSAPLDYLHGHGSLISGLETA
LEGHEVGDKFDVAVGANDAYGQYDENLVQRVPKDVFMGVDELQVGMRFL
AETDQGPVPVEITAVEDDHVVVDGNHMLAGQNLKFNVEVVAIREATEEE
LAHGHVHGAHDHHHDHDHD.

ECOLI FKBP-type 16 kDa peptidyl-prolyl cis-trans isomerase of Escherichia coli (strain K12). FKBP domain, IF domain double underlined.

(SEQ ID NO: 28)
MSESVQSNSAVLVHFTLKLDDGTTAESTRNNGKPALFRLGDASLSEGLEQ
HLLGLKVGDKTTFSLEPDAAFGVPSPDLIQYFSRREFMDAGEPEIGAIML
FTAMDGSEMPGVIREINGDSITVDFNHPLAGQTVHFDIEVLEIDPALEA.

Peptidyl-prolyl cis-trans isomerase of Thermus thermophilus (strain HB8/ATCC 27634) FKBP domain, IF domain double underlined.

(SEQ ID NO: 29)
MKVGQDKVVTIRYTLQVEGEVLDQGELSYLHGHRNLIPGLEEALEGREEG
EAFQAHVPAEKAYGPHDPEGVQVVPLSAFPEDAEVVPGAQFYAQDMEGNP
MPLTVVAVEGEEVTVDFNHPLAGKDLDFQVEVVKVREATPEELLHGHAH.

Analysis of the Q-tag sequences for KalbTG

Analysis of the KalbTG peptide substrates that were identified using labeling assays with a set of peptides comprising 5-mer amino acid sequences with three 3 N- and C-terminal glycine residues (G₁ and G₂) attached revealed a set of characteristics shared by these substrates. Specific examples were as follows:

(SEQ ID NO: 30)
G₁-YRYRQ-G₂,
(SEQ ID NO: 31)
G₁-RYRQR-G₂,
(SEQ ID NO: 32)
G₁-RYSQR-G₂,
(SEQ ID NO: 33)
G₁-FRQRQ-G₂,
(SEQ ID NO: 34)
G₁-RQRQR-G₂,
(SEQ ID NO: 35)
G₁-FRQRG-G₂,
(SEQ ID NO: 36)
G₁-QRQRQ-G₂,
(SEQ ID NO: 37)
G₁-YKYRQ-G₂,
(SEQ ID NO: 38)
G₁-QYRQR-G₂,
(SEQ ID NO: 39)
G₁-YRQTR-G₂,
(SEQ ID NO: 40)
G₁-LRYRQ-G₂,
(SEQ ID NO: 41)
G₁-YRQSR-G₂,
(SEQ ID NO: 42)
G₁-YQRQR-G₂,
(SEQ ID NO: 43)
G₁-RYTQR-G₂,
(SEQ ID NO: 44)
G₁-RFSQR-G₂,
(SEQ ID NO: 45)
G₁-QRQTR-G₂,
(SEQ ID NO: 46)
G₁-WQRQR-G₂,
(SEQ ID NO: 47)
G₁-PRYRQ-G₂,
(SEQ ID NO: 48)
G₁-AYRQR-G₂,
(SEQ ID NO: 49)
G₁-VRYRQ-G₂,
(SEQ ID NO: 50)
G₁-VRQRQ-G₂,
and
(SEQ ID NO: 51)
G₁-YRQRA-G₂.

For the KalbTG, the data revealed that an acyl-donor substrate including a 5-mer amino acid sequence having the formula Xaa₁-Xaa₂-Xaa₃-Xaa₄-Xaa₅, where Xaa is any amino acid, generally complied with several design rules. First, each 5-mer sequence included at least one glutamine (Q). More particularly, at least one of the third, fourth, and fifth positions of the 5-mer sequence (i.e., Xaa₃, Xaa₄, and Xaa₅) was a glutamine. Several sequences were observed that included a glutamine at each of the third and fifth positions. Furthermore, the observation was made that each 5-mer sequence included at least one arginine (R). More particularly, at least one of the fourth and fifth positions of the 5-mer sequence (i.e., Xaa₄ and Xaa₅) was an arginine.

### Labeling Assays

### 1. Fluorescent Dye

For labeling assays, the chaperone SlyD from Thermus thermophilus (Universal Protein Resource (UniProt) Number Q5SLE7) was used as a labeling scaffold for KalbTG. The SlyD sequence is:

(SEQ ID NO: 29)
MKVGQDKVVTIRYTLQVEGEVLDQGELSYLHGHRNLIPGLEEALEGREEG
EAFQAHVPAEKAYGPHDPEGVQVVPLSAFPEDAEVVPGAQFYAQDMEGNP
MPLTVVAVEGEEVTVDFNHPLAGKDLDFQVEVVKVREATPEELLHGHAH.

A KalbTG glutamine donor sequence (Q-tag, underlined) was recombinantly grafted onto the FKBP domain of SlyD, yielding the following polypeptide sequence:

(SEQ ID NO: 52)
MKVGQDKVVTIRYTLQVEGEVLDQGELSYLHGHRNLIPGLEEALEGREEG
EAFQAHVPAEKAYGAGSGGGGRYRQRGGGGGSSGKDLDFQVEVVKVREAT
PEELLHGHAHHHHHHHH.

The 8X-histidine-tagged protein was produced in E. coli B121 Tuner and purified by standard Ni Sepharose-based immobilized metal ion affinity and size exclusion chromatography (HisTrap, Superdex 200; GE Healthcare). All peptides were synthesized via standard Fluorenylmethyloxycarbonyl (FMOC)-based solid phase peptide synthesis.

Labeled peptides were chemically synthesized to have (in order from N-terminus to C-terminus) a Z-protecting group (i.e., a carboxybenzyl group), a transglutaminase lysine donor sequence (K-tag), 8-amino-3,6-dioxaoctanoic acid (020c), peptide, and a Cy3 or Cy5 fluorescent dye. The primary chemical structure of the labeled peptides was:

(SEQ ID NO: 53)
Z-RYESKG-O2Oc-EUEUEUEUEUEUEUEUEUEUEUEUEUEUEUEUEUEU
EUEU-C(sCy3-MH)-OH.

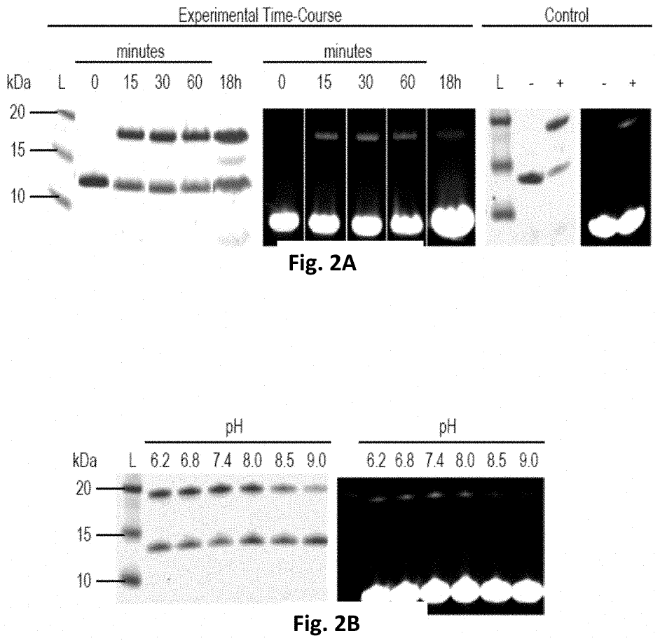

Labeling reactions were performed for 15 minutes at 37° C. in the presence of 72 μM substrate protein, 720 μM label peptide and 1 μM transglutaminase in 200 mM MOPS pH 7.2 and 1 mM EDTA. After incubation for 30 minutes at 37° C., 1 mM K-tag-Cy5 or -Cy3 was added and incubated for an additional 15 minutes at 37° C. The reaction was stopped by the addition of 50 mM TCA. Samples were taken between incubation steps and analyzed by SDS-PAGE, in-gel fluorescence (BioRad ChemiDoc gel documentation system, Cy3 and Cy5 LED and filter sets). Results are shown in FIG. 2.

### 2. Ruthenium Label

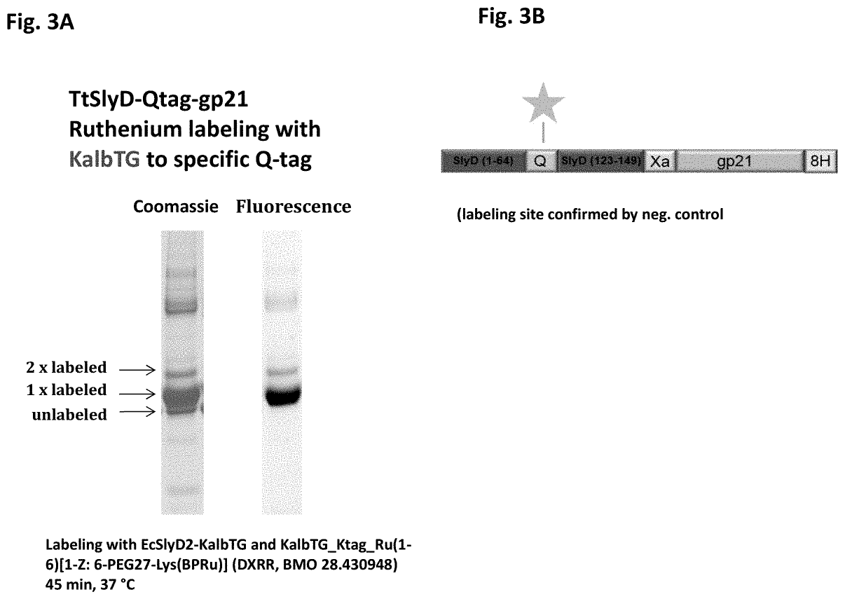

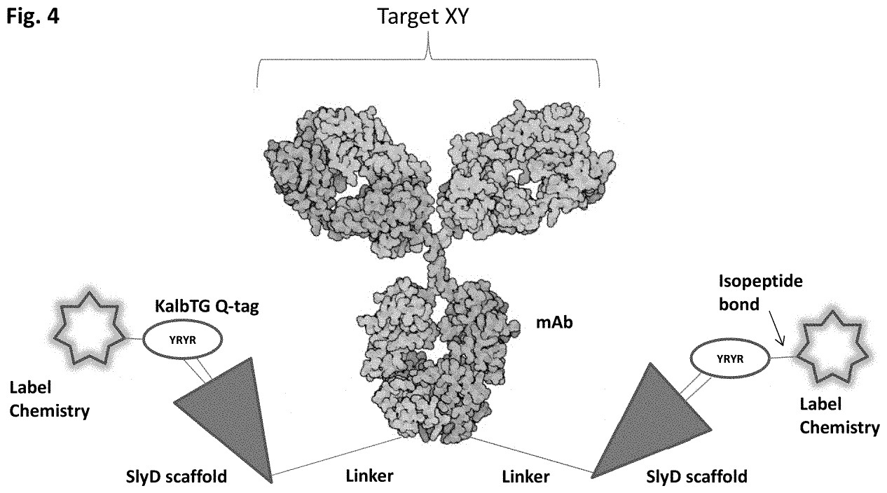

A similar Q-tag construct was tested, consisting of a slyD Q-tag gp21 fusion (see FIG. 3B), wherein the label was a ruthenium-label. The results in SDS-PAGE in FIG. 3A show a good 1:1 ratio of label to construct. FIG. 3 as a whole shows TtSlyD-Qtag-gp21 Ruthenium labeling with KalbTG to specific Q-tag (YRYRQ).

### Production of Biotin- and Ruthenium Labeled TtSlyD-Gp21 (HTLV) Antigens

For further labeling assays, a recombinant fusion between the chaperone SlyD from Thermus thermophilus (Universal Protein Resource (UniProt) Number Q5SLE7), the HTLV viral envelope glycoprotein ‘gp21’ (Genbank Accession Number DQ224032.1) and, in one example, the chaperone SlpA from Escherichia coli (UniProt Number P0AEM0) was used as a labeling scaffold for MTG or KalbTG. The SlyD sequence is:

(SEQ ID NO: 54)
MKVGQDKVVTIRYTLQVEGEVLDQGELSYLHGHRNLIPGLEEALEGREEG
EAFQAHVPAEKAYGPHDPEGVQVVPLSAFPEDAEVVPGAQFYAQDMEGNP
MPLTVVAVEGEEVTVDFNHPLAGKDLDFQVEVVKVREATPEELLHGHAH

A MTG or KalbTG glutamine donor sequence (Q-tag) was recombinantly grafted onto the FKBP domain of SlyD, yielding the following polypeptide sequences:

SlyD with MTG Q-tag:

(SEQ ID NO: 55)
MKVGQDKVVTIRYTLQVEGEVLDQGELSYLHGHRNLIPGLEEALEGREEG
EAFQAHVPAEKAYGAGSGGGGDYALQGGGGGSSGKDLDFQVEVVKVREAT
PEELLHGHAH

SlyD with KalbTG Q-tag:

(SEQ ID NO: 56)
MKVGQDKVVTIRYTLQVEGEVLDQGELSYLHGHRNLIPGLEEALEGREEG
EAFQAHVPAEKAYGAGSGGGGYRYRQGGGGGSSGKDLDFQVEVVKVREAT
PEELLHGHAH

The SlpA sequence is:

(SEQ ID NO: 57)
MSESVQSNSAVLVHFTLKLDDGTTAESTRNNGKPALFRLGDASLSEGLEQ
HLLGLKVGDKTTFSLEPDAAFGVPSPDLIQYFSRREFMDAGEPEIGAIML
FTAMDGSEMPGVIREINGDSITVDFNHPLAGQTVHFDIEVLEIDPALEA

The main HTLV antigen and viral envelope glycoprotein sequence ‘gp21’ is:

(SEQ ID NO: 58)
IVSSACNNSLILPPFSLSPVPTVGSRSRRAVPVAVWFVSALAMGAGVAGG
ITGSMSLASGKSLLHEVDKDISQLTQAIVKNHKNLLKIAQYAAQNRRGLD
LLFWEQGGLCKALQEQCCFLNITNSHVSILQERPPLENRVLTGWGLNWDL
GLSQWAREALQTGITLVALLLLVILAGPCIRCPCRTMHP

The gp21 ectodomain polypeptide sequence, engineered for better solubility, stability and reactivity in the immunoassay, is:

(SEQ ID NO: 59)
MSLASGKSLLHEVDKDISQLTQAIVKNHKNLLKIAQYAAQNRRGLDLLFW
EQGGLAKALQEQAAFLNITNSHVSILQERPPLENRVLTGWGLNWDLGLSQ
WAREALQTG

The recombinant fusion sequences used for the labeling assays were:

‘TtSlyQD-Xa-gp21-8H’:
(SEQ ID NO: 60)
MKVGQDKVVTIRYTLQVEGEVLDQGELSYLHGHRNLIPGLEEALEGREEG
EAFQAHVPAEKAYGAGSGGGGDYALQGGGGGSSGKDLDFQVEVVKVREAT
PEELLHGHAHGGGSGGGSGGGSGGGSGGGSGGGIEGRMSLASGKSLLHEV
DKDISQLTQAIVKNHKNLLKIAQYAAQNRRGLDLLFWEQGGLAKALQEQA
AFLNITNSHVSILQERPPLENRVLTGWGLNWDLGLSQWAREALQTGGHHH
HHHHH
‘TtSlyD-Xa-gp21-8H’:
(SEQ ID NO: 61)
MKVGQDKVVTIRYTLQVEGEVLDQGELSYLHGHRNLIPGLEEALEGREEG
EAFQAHVPAEKAYGPHDPEGVQVVPLSAFPEDAEVVPGAQFYAQDMEGNP
MPLTVVAVEGEEVTVDFNHPLAGKDLDFQVEVVKVREATPEELLHGHAHG
GGSGGGSGGGSGGGSGGGSGGGIEGRMSLASGKSLLHEVDKDISQLTQAI
VKNHKNLLKIAQYAAQNRRGLDLLFWEQGGLAKALQEQAAFLNITNSHVS
ILQERPPLENRVLTGWGLNWDLGLSQWAREALQTGGHHHHHHHH
‘TtSlyKQD-SlpA-Xa-gp21-8H’:
(SEQ ID NO: 62)
MKVGQDKVVTIRYTLQVEGEVLDQGELSYLHGHRNLIPGLEEALEGREEG
EAFQAHVPAEKAYGAGSGGGGYRYRQGGGGGSSGKDLDFQVEVVKVREAT
PEELLHGHAHGGGSGGGSGGGSGGGSGGGSGGGMSESVQSNSAVLVHFTL
KLDDGTTAESTRNNGKPALFRLGDASLSEGLEQHLLGLKVGDKTTFSLEP
DAAFGVPSPDLIQYFSRREFMDAGEPEIGAIMLFTAMDGSEMPGVIREIN
GDSITVDFNHPLAGQTVHFDIEVLEIDPALEAGGGSGGGSGGGSGGGSGG
GSGGGIEGRMSLASGKSLLHEVDKDISQLTQAIVKNHKNLLKIAQYAAQN
RRGLDLLFWEQGGLAKALQEQAAFLNITNSHVSILQERPPLENRVLTGWG
LNWDLGLSQWAREALQTGGHHHHHHHH

The 8X-histidine-tagged proteins were produced in E. coli B121 Tuner and purified by standard Ni Sepharose-based immobilized metal ion affinity and size exclusion chromatography (HisTrap, Superdex 200; GE Healthcare).

Labeled peptides were chemically synthesized to have (in order from N-terminus to C-terminus) a “Z-” group (i.e., a carboxybenzyl group), a transglutaminase lysine donor sequence (K-tag), polyethylene glycol (PEG), peptide, and a Biotin label or Bipyrimidine Ruthenium (BPRu) complex. The primary chemical structures of the labeled peptides were:

KalbTG K-tag-Bi (“Bi” represents a Biotin label):

Z-RYESKG-PEG27-K(Bi)—OH (2532.0 g/mol)

KalbTG K-tag-Ru (“Ru” represents a Ruthenium-based label for electrochemoluminescence): Z-RYESKG-PEG27-K(BPRu)-OH (2958.4 g/mol)

If not noted otherwise, typical labeling reactions were performed for 15 minutes at 37° C. in the presence of 65 μM substrate protein, 1000 μM label peptide and 0.1 μM transglutaminase in 200 mM MOPS pH 7.4 for labeling with the KalbTG enzyme and at 37° C. in the presence of 72 μM substrate protein, 720 μM label peptide and 1 μM transglutaminase in 200 mM MOPS pH 7.4 with the MTG enzyme. Transglutaminase labeling is described in more detail in applications US 2016/0178627 ‘System and Method for Identification and Characterization of Transglutaminase Species’ and WO 2016/096785 ‘IDENTIFICATION OF TRANSGLUTAMINASE SUBSTRATES AND USES THEREFOR’.

The labeling reaction mix was separated by size exclusion chromatography (Superdex 200; GE Healthcare) and fractions containing labeled fusion proteins were isolated for subsequent analysis.

### Analysis of Biotin- and Ruthenium Labeling and Immunoassay

The quality and quantity of labeling reactions were analyzed by SDS-PAGE. Efficiency of Biotin labeling was estimated by the shift in molecular weight of the bands in the coomassie-stained gel. Efficiency of Ruthenium labeling was estimated by the shift in molecular weight of the bands in the coomassie-stained gel and by in-gel fluorescence (BioRad ChemiDoc gel documentation system, Cy3 LED and filter set). Diagnostic immunoassays (Roche HTLV I/II) were performed according to the manufacturer's instructions on an Elecsys e411 instrument, replacing the biotin labeled antigen moiety in reagent R1 of the test by ‘TtSlyD-Xa-gp21-8H’ biotin labeled with MTG or ‘TtSlyKQD-SlpA-Xa-gp21-8H’ biotin labeled with KalbTG, respectively, and replacing the Ruthenium labeled antigen moiety in reagent R2 of the test by ‘TtSlyD-Xa-gp21-8H’ Ruthenium labeled with MTG or ‘TtSlyKQD-SlpA-Xa-gp21-8H’ Ruthenium labeled with KalbTG, respectively. Postive controls were performed with HTLV positive sera and the commercially available HTLV I/II kit. Negative controls were performed with HTLV negative sera and with HTLV positive sera in combination with different calibrator and control solutions.

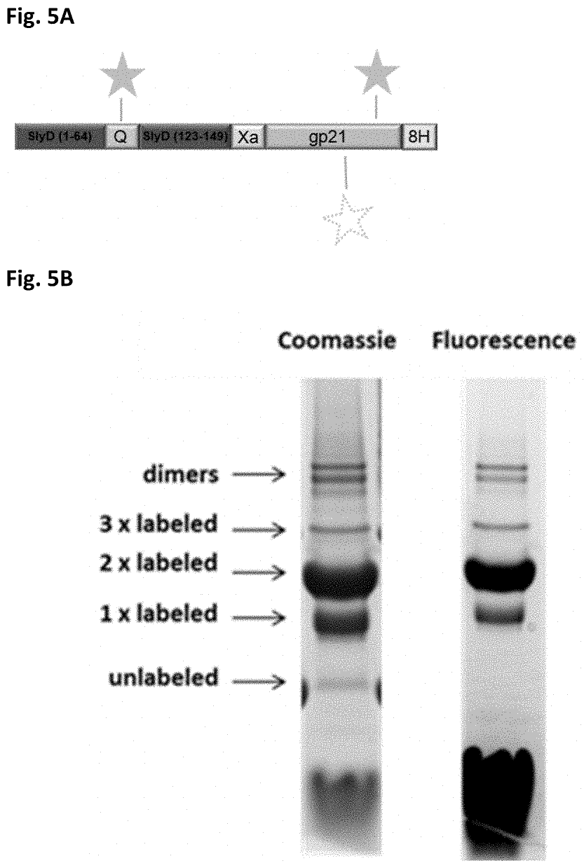

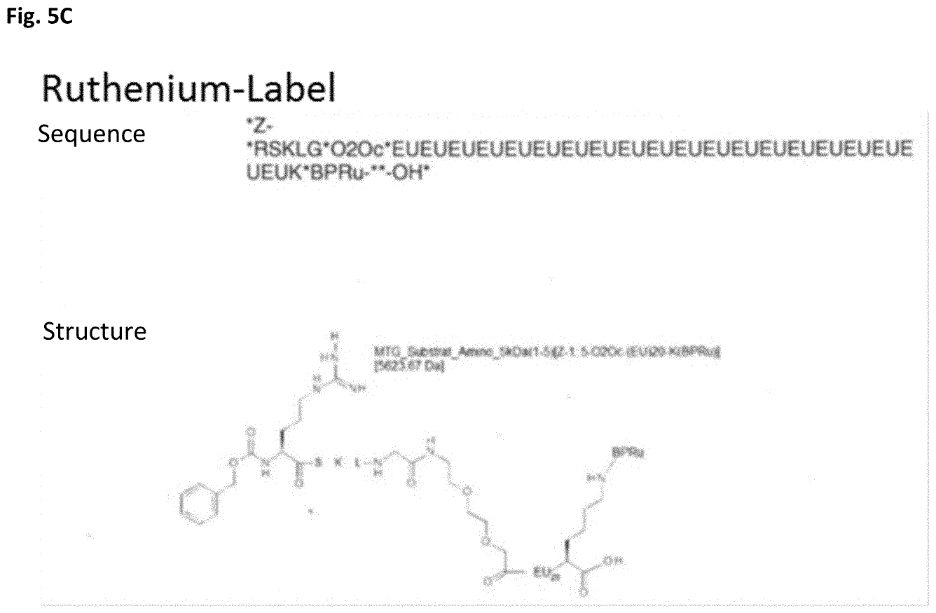

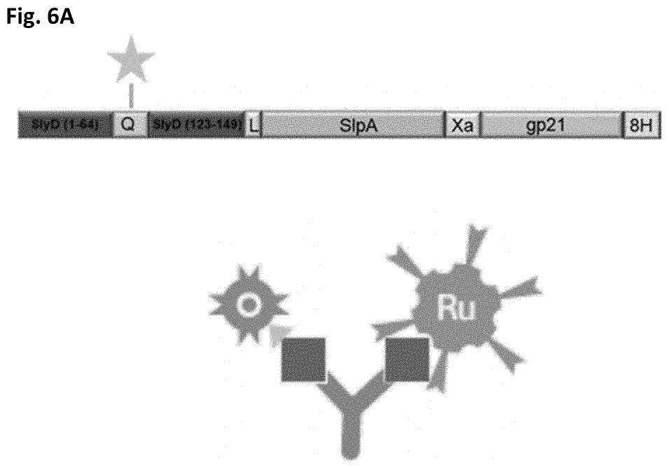

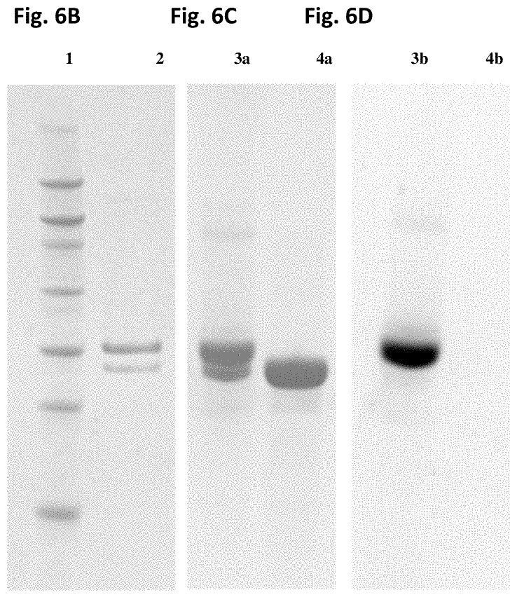

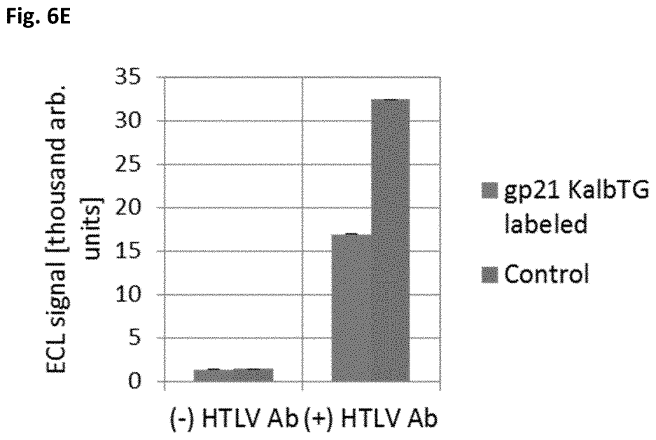

For results, see FIGS. 5 and 6.

### Mass Spectrometric Analysis of TtSlyQD-Xa-gp21-8H Ruthenium Labeling

FIG. 5A schematically depicts the fusion protein ‘TtSlyQD-Xa-gp21-8H’ of SEQ ID NO:60. FIG. 5B shows results after SDS-PAGE analysis showing specific and unspecific Ruthenium labeling of the recombinant gp21 (HTLV) antigen with the bacterial transglutaminase of Streptomyces mobaraensis. Depicted are unlabeled, single-labeled, double-labeled, and triple-labeled ‘TtSlyQD-Xa-gp21-8H’ fusion proteins after Ru labeling. Ru-label see in FIG. 5C.

Two pooled fractions of the Ruthenium labelled fusion protein were analyzed, [1] TtSlyQD-Xagp21-8H_Fraction 17-19 and [2] TtSlyQDXa-gp21-8H_Fraction 20-23; unlabeled ‘TtSlyQD-Xagp21-8H’ protein served a control (TtSlyQD-Xa-gp21-8H_Control).

Peptide mapping was performed. After peptide mapping by digestion with Trypsin, chromatographic separation, subsequent MS/MS analysis, and database searching in combination with manual interpretation tools, the following was found:

(a) The tryptic peptide ‘62-85’ “AYGAGSGGGGDYALQGGGGGSSGK” SEQ ID NO:63 (numbering is referred to the sequence given below) with one label was found in both fractions ‘TtSlyQD-Xa-gp21-8H_Fraction 17-19’ and ‘TtSlyQD-Xa-gp21-8H_Fraction 20-23’.

(b) The tryptic peptide ‘195-222’ “ALQEQAAFLNITNSHVSILQERPPLENR” SEQ ID NO:64 with one label was found in traces only in ‘TtSlyQD-Xa-gp21-8H_Fraction 17-19’.

(c) The tryptic peptide ‘241-255’ “EALQTGGHHHHHHHH” SEQ ID NO:65 (His-Tag) with one label was found in both fractions ‘TtSlyQD-Xa-gp21-8H_Fraction 17-19’ and ‘TtSlyQD-Xagp21-8H_Fraction 20-23’.

(d) In addition to the above, also other Ruthenium labelled peptides/byproducts were found to be present in both Fractions, however in insignificant quantities.

Amino acid sequence of TtSlyQD-Xa-gp21-8H, tryptic peptides as given above in (a-c) are marked in boldface and underlined:

(SEQ ID NO: 60)
MKVGQDKVVTIRYTLQVEGEVLDQGELSYLHGHRNLIPGLEEALEGREEG
EAFQAHVPAEKAYGAGSGGGGDYALQGGGGGSSGKDLDFQVEVVKVREAT
PEELLHGHAHGGGSGGGSGGGSGGGSGGGSGGGIEGRMSLASGKSLLHEV
DKDISQLTQAIVKNHKNLLKIAQYAAQNRRGLDLLFWEQGGLAKALQEQA
AFLNITNSHVSILQERPPLENRVLTGWGLNWDLGLSQWAREALQTGGHHH
HHHHH

Recombinant gp21 (HTLV) antigen, site-specifically labeled with Biotin and Ruthenium using the bacterial transglutaminase of Kutzneria albida demonstrates the advantages of the invention in the Elecsys HTLV-I/II in-vitro diagnostic assay.

The results are presented in FIG. 6 and the following.

FIG. 6 A depicts schematic representations showing the primary structure of the Ruthenium and Biotin labeled gp21 antigen (Q=KalbTG Q-tag, L=linker, Xa=factor Xa cleavage site, 8H=His-tag) and the diagnostic test principle; Serum Antibody from a patient sample (red) is bridging Biotin and Ruthenium labeled antigens (green).

FIG. 6 B-D: SDS-PAGE analysis showing single Biotin or Ruthenium labeling of gp21 with KalbTG. B: Coomassie stained SDS-PAGE gel showing Biotin Labeling, lane1: prestained protein molecular weight marker, lane 2: TtSlyKQD-SlpA-Xa-gp21-8H incubated with KalbTG and biotin label. C: Coomassie stained SDS-PAGE gel showing Ruthenium labeling, lane 3a: TtSlyKQDSlpA-Xa-gp21-8H incubated with KalbTG and Ruthenium label, lane 4a: Unmodified TtSlyKQDSlpA-Xa-gp21-8H (control). D: Fluorescence image of the SDS-PAGE analysis shown in C. Lanes 3b-4b correspond to lanes 3a-4a.

FIG. 6 E: Elecsys assay (HTLV-I/II) on sera negative (left) and positive (right) for HTLV antibody using the MTG labeled reagents or the commercial kit (control) reagents. Note the positive control signal is higher than KalbTG labeled gp21 signal, since the labeling stoichiometry in the control is higher than 1:1. That is to say, the KalbTG labeled molecule carries less label. Measurements were performed in duplicates, shown is the average signal.

The amino acid sequence of TtSlyKQD-SlpA-Xa-gp21-8H is as follows:

(SEQ ID NO: 62)
MKVGQDKVVTIRYTLQVEGEVLDQGELSYLHGHRNLIPGLEEALEGREEG
EAFQAHVPAEKAYGAGSGGGGYRYRQGGGGGSSGKDLDFQVEVVKVREAT
PEELLHGHAHGGGSGGGSGGGSGGGSGGGSGGGMSESVQSNSAVLVHFTL
KLDDGTTAESTRNNGKPALFRLGDASLSEGLEQHLLGLKVGDKTTFSLEP
DAAFGVPSPDLIQYFSRREFMDAGEPEIGAIMLFTAMDGSEMPGVIREIN
GDSITVDFNHPLAGQTVHFDIEVLEIDPALEAGGGSGGGSGGGSGGGSGG
GSGGGIEGRMSLASGKSLLHEVDKDISQLTQAIVKNHKNLLKIAQYAAQN
RRGLDLLFWEQGGLAKALQEQAAFLNITNSHVSILQERPPLENRVLTGWG
LNWDLGLSQWAREALQTGGHHHHHHHH.

1. A recombinant transglutaminase (TG) substrate according to the following general formula I
(F*-L)_{y}-X (I)
wherein
F* is selected from an amino acid sequence of the FKBP domain of an FKBP polypeptide, wherein the “insert-in-flap” (IF) domain thereof is, at least in part, replaced by an amino acid sequence (“Q-tag”) of 5 to 20 amino acids, the Q-tag comprising a sub-sequence of 5 contiguous amino acids having at least 80% sequence identity to the YRYRQ portion of the peptide sequence X₁-YRYRQ-X₂ (SEQ ID NO. 1), wherein X₁ and X₂ are absent or constitute linker amino acids;
L is absent or is selected from a linker amino acid sequence; and
X is a protein of interest;
y is an integer of between 1 and 100,
and wherein said TG substrate is a substrate for the TG function of the Kutzneria albida TG according to SEQ ID No. 23.

2. The recombinant transglutaminase (TG) substrate according to claim 1, wherein said FKBP domain is selected from a eukaryotic or bacterial FKBP polypeptide selected from FKBP12, AIP, AIPL1, FKBP1A, FKBP1B, FKBP2, FKBP3, FKBP5, FKBP6, FKBP7, FKBP8, FKBP9, FKBP9L, FKBP10, FKBP11, FKBP14, FKBP15, FKBP52, LOC541473, and SLYD, and homologs of the FKBP domains thereof.

3. The recombinant transglutaminase (TG) substrate according to claim 1, wherein said FKBP domain comprises between about 120 to 170 of the N-terminal amino acids of said FKBP polypeptide.

4. The recombinant transglutaminase (TG) substrate according to claim 1, wherein said FKBP domain comprises the N-terminal amino acids 1 to 64 and 123 to 149 of the SLYD polypeptide, and wherein amino acids 65 to 122 are replaced by said Q-tag.

5. The recombinant transglutaminase (TG) substrate according to claim 1, wherein said linker sequence L comprises between 1 to 20 amino acids, wherein said amino acids do not interfere essentially with the FKBP domain and/or the protein of interest.

6. The recombinant transglutaminase (TG) substrate according to claim 1, wherein said protein of interest is selected from an enzyme, an antigen, such as a viral protein, an antibody or fragment thereof, and other immunological binding partners.

7. An in vitro method for labelling a protein of interest, comprising a) providing the recombinant transglutaminase (TG) substrate according to claim 1 being attached to a protein of interest,
b) providing an effective amount of the transglutaminase of Kutzneria albida, according to SEQ ID No. 23,
c) providing a suitable label linked comprising an alkyl-amine group, and
d) contacting said components according to a) to c), whereby said transglutaminase attaches said label to said substrate.

8. The method according to claim 7, wherein said transglutaminase of Kutzneria albida is recombinantly produced.

9. The method according to claim 7, wherein said label is selected from an enzyme, biotin, a radioactive group, a dye, an isotope, a chemiluminescent label, and a metal.

10. The method according to claim 7, wherein said labeling is achieved in a stoichiometric ratio of label and protein of interest at about 1:1.

11. The method according to claim 7, wherein said protein of interest is selected from an enzyme, an antigen, a viral protein, an antibody or fragment thereof, and other immunological binding partners.

12. A pharmaceutical or diagnostic composition comprising at least one labeled protein of interest as produced according to a method according to claim 7, together with pharmaceutically acceptable carrier compounds.

13. The pharmaceutical or diagnostic composition according to claim 12, wherein said protein of interest is labelled at a stoichiometric ratio of label and protein of interest of about 1:1.
